(19) United States
(12) Patent Application Publication
(10) Pub. No.: US 2015/0018616 A1
(43) Pub. Date: Jan. 15, 2015

(54) MEDICAL TREATMENT INSTRUMENT

(71) Applicant: Terumo Kabushiki Kaisha, Tokyo (JP)

(72) Inventor: Kenichi KUMOYAMA, Fujinomiya-shi (JP)

(21) Appl. No.: 14/500,501

(22) Filed: Sep. 29, 2014

Related U.S. Application Data
(63) Continuation of application No. PCT/JP2013/056536, filed on Mar. 8, 2013.

(30) Foreign Application Priority Data
Mar. 28, 2012 (JP) 2012-072773

(51) Int. Cl. A61M 25/00; A61B 1/05; A61M 29/00

(52) U.S. Cl. CPC A61M 25/0068; A61M 29/00; A61M 25/008; A61B 1/05; A61M 2025/0081; A61M 2210/0681
USPC 600/109; 604/264; 606/199

(57) ABSTRACT

A medical treatment instrument includes a tube-shaped elongated main body; and an insertion guide part mounted to a distal end of the elongated main body. The insertion guide part includes a frame body mounted to a distal portion of the elongated main body; and a flexible bridge portion which is disposed so as to bridge points of an end edge of the frame body, the flexible bridge portion including a linear body or band-shaped body projecting in a distal direction beyond the elongated main body.

### CROSS-REFERENCE TO RELATED APPLICATIONS

This application is a continuation application filed under 35 U.S.C. 111(a) claiming the benefit under 35 U.S.C. §§120 and 365(c) of PCT International Application No. PCT/JP2013/056536 filed on Mar. 8, 2013, which is based upon and claims the benefit of priority of Japanese Application No. 2012-072773 filed on Mar. 28, 2012, the entire contents of which are hereby incorporated by reference in their entireties.

### BACKGROUND

1. Technical Field

The present disclosure relates to a medical treatment instrument.

2. Background Art

Conventionally, there have been known medical treatment instruments, such as endoscopes and catheters (see, for example, JP-T-2008-526360).

The medical treatment instrument described in JP-T-2008-526360 includes an elongated insertion section having an imaging device and a fluid conveyance lumen, and is so configured that in the state in which the insertion section is inserted in the body of a patient, observation can be performed based on an image captured by the imaging device, and a fluid such as water mixed with a drug can be conveyed into the inside of the body.

However, the medical treatment instrument according to the related art such as the one described in JP-T-2008-526360 has a problem; because a distal end surface of the insertion section has a steeply rising shape, the distal end of the insertion section is easily caught in a living body, causing difficulty inserting the medical treatment instrument into a living body.

### SUMMARY OF INVENTION

An objective of certain embodiments of the present invention is to provide a medical treatment instrument that can be easily inserted into a living body.

According to one embodiment of the present invention, a medical treatment instrument includes: a tube-shaped elongated main body; and an insertion guide part mounted to a distal end of the elongated main body, in which the insertion guide part includes a frame body mounted to a distal portion of the elongated main body, and a flexible bridge portion which is bridgingly provided between points of an end edge of the frame body and which is composed of a linear body or band-shaped body projecting toward a distal side of the elongated main body.

According to one embodiment of the present invention, the medical treatment instrument includes the insertion guide part which has the bridge portion bridgingly provided between points of the frame body mounted to a distal portion of the elongated main body. This ensures that the elongated main body can be guided while forcing open an insertion route toward the outer circumference of the elongated main body by the bridge portion. Therefore, the distal end of the elongated main body can be prevented from being caught in a living body. In addition, because the bridge portion is composed of a flexible linear body or band-shaped body, the thickness size of the bridge portion as viewed from the insertion direction can be made small, and the bridge portion can follow the shape of the insertion route through deformation. Therefore, the ability of the insertion guide part to pass the insertion route can be enhanced, while suppressing the resistance at the time of insertion. Accordingly, the medical treatment instrument can be inserted easily.

In one aspect, a plurality of the bridge portions are provided in different positions of the end edge, and distal ends in a projection direction of the bridge portions are not fixed to each other.

In one aspect, the plurality of bridge portions are provided in different positions of the end edge of the frame body. This ensures that the distal end of the elongated main body can be guided at a plurality of parts, and the distal end of the elongated main body can be prevented from being caught. Further, because the distal ends in the projection direction of the bridge portions are not fixed to each other, the bridge portions can be deformed flexibly. Accordingly, the medical treatment instrument can be inserted more easily.

In one aspect, the bridge portion is composed of a band-shaped body, and an end portion in a width direction of the band-shaped body projects toward a distal side of the elongated main body.

In one aspect, the bridge portion is composed of a band-shaped body, and an end portion in the width direction of the band-shaped body is projecting toward the distal side. This ensures that a degree of rigidity of the bridge portion necessary for guiding can be secured, while making keeping the bridge portion thin as viewed from the insertion direction. Therefore, the bridge portion can be prevented from being collapsed toward the proximal side of the elongated main body, attendant on insertion. Accordingly, guiding by the insertion guide part can be performed reliably.

In one aspect, the insertion guide part decreases in diameter in multiple steps along a distal direction.

In one aspect, the insertion guide part decreases in diameter in multiple steps along the distal direction, so that the insertion guide part has a kind of link structure whose centers are determined by junction points between the steps. Therefore, a degree of rigidity of the insertion guide part necessary for guiding can be secured, while maintaining a follow-up performance of the insertion guide part in relation to an insertion route. In addition, because the degree of decrease in diameter can be set on the basis of each of the steps, the projection shape of the insertion guide part can be set according to the use of the medical treatment instrument or the insertion route. This enables further easier insertion of the medical treatment instrument.

In one aspect, the medical treatment instrument includes an image information acquisition section which is provided inside the elongated main body and which acquires an image of a forward side in an insertion direction of the insertion guide part.

In one aspect, the medical treatment instrument is provided with the image information acquisition section which acquires an image of the forward side in the insertion direction of the insertion guide part, so that image information on the insertion guide part and the forward side of the insertion guide part can be acquired. This enables the operator to operate the medical treatment instrument while checking the image information acquired. In addition, because the bridge portion of the insertion guide part is composed of a linear body or band-shaped body, the area of projection in the insertion direction of the bridge portion can be made small. Therefore, the bridge portion can be prevented from obstructing the viewing of the image acquired by the image information acquisition section. Accordingly, a sufficient field of view for the image information acquisition section can be secured.

In one aspect, the medical treatment instrument includes a fluid conveyance path which is provided in the elongated main body and which is open toward the insertion guide part.

In one aspect, the fluid conveyance path opening toward the insertion guide part is provided, so that it is possible to convey a fluid from the inside of a living body to the outside or to convey a fluid from the outside into the inside of the living body, through the fluid conveyance path. Because the bridge portion of the insertion guide part is composed of a linear body or band-shaped body, the bridge portion can be prevented from blocking the conveyance of a fluid.

In one aspect, the medical treatment instrument includes an expansion body which is provided at an outer circumference on a distal side of the elongated main body and which is expanded outward in a radial direction of the elongated main body.

In one aspect, the expansion body can be assuredly guided into a stenosed part in a living body by the insertion guide part. Consequently, the stenosed part can be expanded and treated by expansion of the expansion body.

In one aspect, the medical treatment instrument is a sinusitis treatment instrument.

In one aspect, where the medical treatment instrument is a sinusitis treatment instrument, it has the insertion guide part. This ensures that the medical treatment instrument can be assuredly inserted into a paranasal sinus and into a natural ostium through which the paranasal sinus and a nasal cavity communicate with each other. Accordingly, a treatment necessary for the sinusitis treatment can be carried out without performing a surgical procedure.

### BRIEF DESCRIPTION OF DRAWINGS

FIG. 1 is a side view showing, partly in section, a medical treatment instrument according to a first embodiment of the present invention.

FIG. 2A is a partial sectional view of a grasping portion of the medical treatment instrument.

FIG. 2B is a partial sectional view of the grasping portion of the medical treatment instrument shown in FIG. 2A from a different perspective.

FIG. 3 is an exploded perspective view of an insertion tube of the medical treatment instrument.

FIG. 4A is an end-side view showing a valve body of the medical treatment instrument.

FIG. 4B is an end view showing the valve body of the medical treatment instrument.

FIG. 5 is a side-end perspective view showing the configuration in the surroundings of an insertion guide part of the medical treatment instrument.

FIG. 6A is an end view showing an insertion guide part of a medical treatment instrument according to a second embodiment of the present invention.

FIG. 6B is a side view showing the insertion guide part of the medical treatment instrument according to the second embodiment of the present invention.

FIG. 7A is an end view showing an insertion guide part of a medical treatment instrument according to a third embodiment of the present invention.

FIG. 7B is a side view showing the insertion guide part of the medical treatment instrument according to the third embodiment of the present invention.

FIG. 8A is an end view showing an insertion guide part of a medical treatment instrument according to a fourth embodiment of the present invention.

FIG. 8B is a side view showing the insertion guide part of the medical treatment instrument according to the fourth embodiment of the present invention.

FIG. 9A is an end view showing an insertion guide part of a medical treatment instrument according to a fifth embodiment of the present invention.

FIG. 9B is a side view showing the insertion guide part of the medical treatment instrument according to the fifth embodiment of the present invention.

FIG. 10 is a view showing a medical treatment instrument according to a modification of the present invention.

### DETAILED DESCRIPTION

Now, embodiments of the present invention will be described below, based on the drawings.

It is to be noted that in a second embodiment and thereafter, component members which are the same as the component members in a first embodiment to be described below and component members which have functions equivalent or similar to the functions of the component members in the first embodiment will be denoted by the same reference symbols as used for the component members in the first embodiment, and descriptions of them will be omitted or simplified.

### First Embodiment

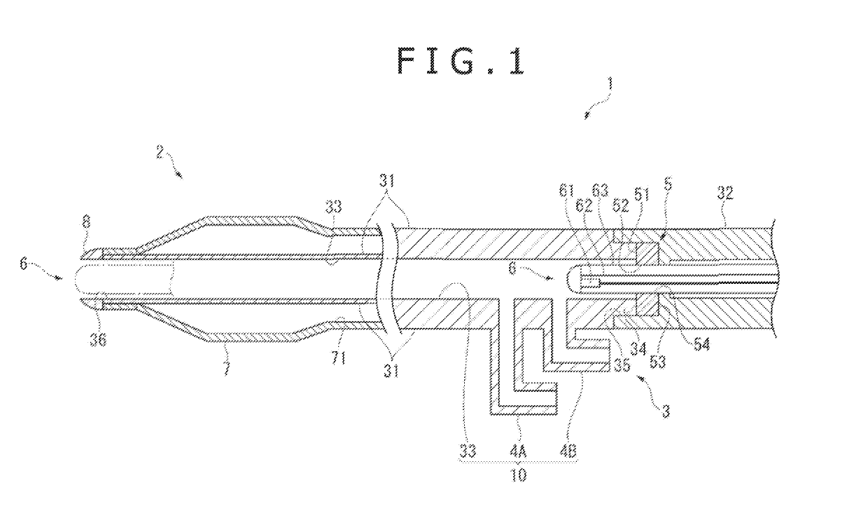

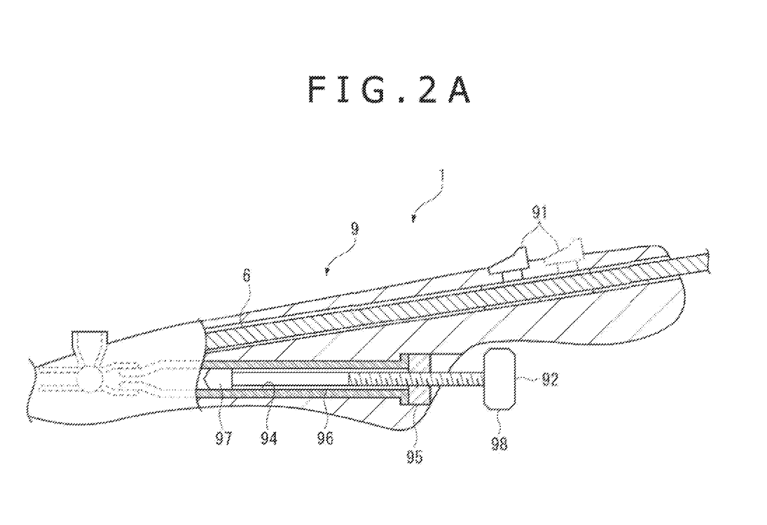

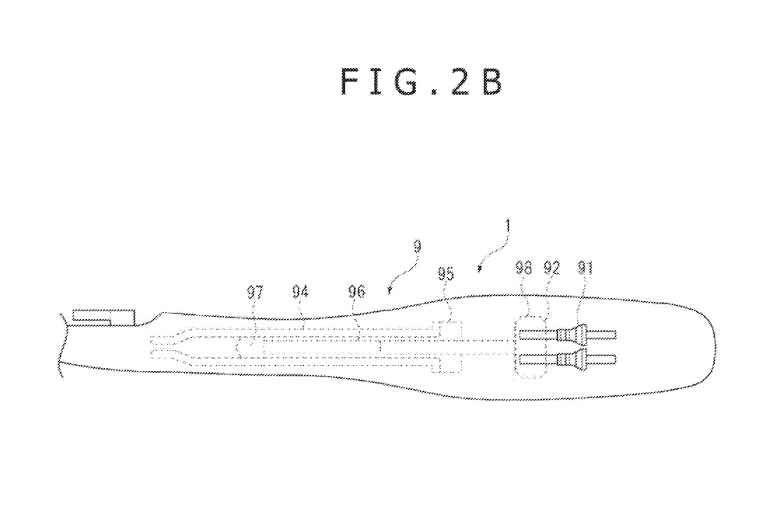

As shown in FIGS. 1, 2A and 2B, a medical treatment instrument 1 according to a first embodiment includes: an insertion section 2 (FIG. 1) that is inserted in a body of a patient; and a grasping portion 9 (FIGS. 2A and 2B) that is provided on a proximal side of the insertion section 2.

In FIG. 1, the insertion section 2 includes: an insertion tube 3 as an elongated main body; first and second conveyance paths 4A and 4B through which to convey an object to be conveyed located within the insertion tube 3, the first and second conveyance paths 4A and 4B being provided at intermediate portions of the insertion tube 3 so as to communicate with the insertion tube 3; a valve body 5 that seals the proximal side of the insertion tube 3, the valve body 5 being provided on the proximal side of the insertion tube 3 relative to the conveyance paths 4A and 4B; an endoscope 6 as an image information acquisition section that acquires an image of the forward side in an insertion direction, the endoscope 6 being inserted in a withdrawable manner in the insertion tube 3; an expansion body 7 that is expanded outward in the radial direction of the insertion tube 3, the expansion body 7 being provided at an outer circumference of the insertion tube 3; and an insertion guide part 8 detachably attached to a distal end of the insertion tube 3.

The insertion tube 3 includes: a distal tube 31 to which the insertion guide part 8 is attached; a proximal tube 32 connected to the grasping portion 9; and a lumen 33 opening toward the insertion guide part 8. The distal tube 31 and the proximal tube 32 are connected to each other in an attachable and detachable manner. The space inside the distal tube 31 and the space inside the proximal tube 32 constitute the lumen 33.

The distal tube 31 is provided with a proximal portion 34 reduced in diameter as compared with the distal side, and is provided with the conveyance paths 4A and 4B which are communicate with the lumen 33. Here, the first conveyance path 4A is a conveyance path through which a fluid is conveyed from the inside of a living body to the outside; specifically, the first conveyance path 4A is so configured that such a fluid as snivel can be sucked therethrough by a suction device (not shown) connected to the first conveyance path 4A. In addition, the second conveyance path 4B is a conveyance path through which a fluid is conveyed into the inside of a living body from the outside; specifically, the second conveyance path 4B is so configured that such a fluid as washing water can be conveyed into the inside of the living body by means of a pump (not shown) connected to the second conveyance path 4B. The first conveyance path 4A, the second conveyance path 4B, and the lumen 33 constitute a fluid conveyance path 10.

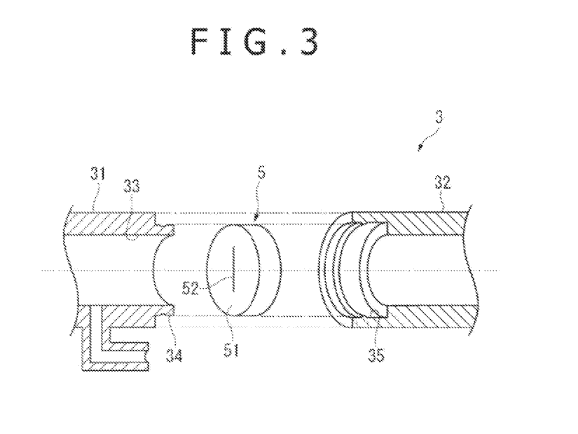

As shown in FIG. 3, the proximal tube 32 is provided, at an end face on the distal side thereof, with a recess 35 greater in inside diameter than the lumen 33, and the valve body 5 is accommodated in the recess 35. In a state wherein the valve body 5 is accommodated in the recess 35, the proximal portion 34 of the distal tube 31 is fitted in the recess 35, whereby the distal tube 31 and the proximal tube 32 are fixed to each other.

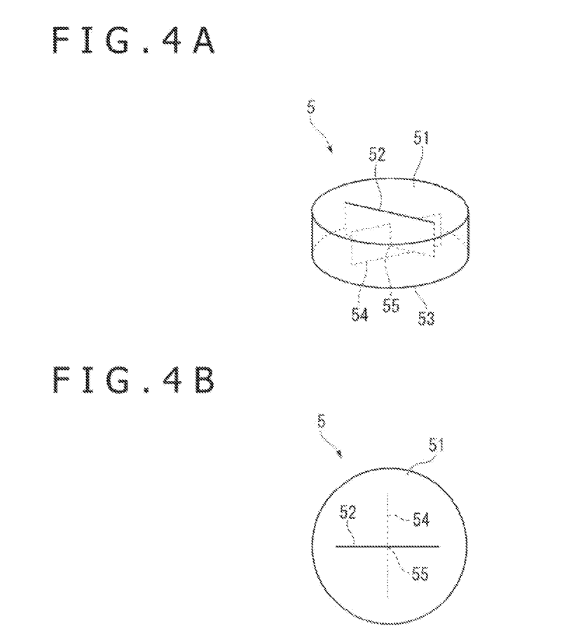

The valve body 5 is configured by use of an elastic material such as rubber or resin. As shown in FIGS. 4A and 4B, the valve body 5 is provided with a first cut 52 formed in an end face 51 on a first side, and with a second cut 54 formed also in an end face 53 on a second side. The first cut 52 and the second cut 54 intersect each other at an intersection portion 55 inside the valve body 5. This configuration enables the valve body 5 to permit insertion and passage of the endoscope 6 through the first cut 52, the intersection portion 55, and the second cut 54 under elastic deformation of the valve body 5, and to hold liquid-tight the lumen 33 of the insertion tube 3. It is to be noted that the intersection angle between the first and second cuts 52 and 54 may not necessarily be a right angle; in other words, the first and second cuts 52 and 54 may intersect each other obliquely.

The endoscope 6 includes: an imaging section 61 having an imaging element such as a CCD (Charge Coupled Device) image sensor; a light transmission section 62, such as an optical fiber, through which to transmit light toward a distal portion of the endoscope 6; and an outer tube 63 in which to accommodate the imaging section 61 and the light transmission section 62. The endoscope 6 may be configured to be able to be curved by operating means such as wire (not shown) connected to a distal portion of the endoscope 6. The imaging section 61 is not restricted to this one configuration; for example, the imaging section 61 may be a digital video camera that uses other imaging element such as a CMOS (Complementary Metal Oxide Semiconductor) image sensor, an image fiber that acquires and transmits images by utilizing optical fibers, or an imaging system that transmits images by an optical system including an objective lens and a plurality of relay lenses.

The expansion body 7 is configured by using a flexible material such as a polymer. The inside of the expansion body 7 communicates with a flow path 71 provided in the periphery of the insertion tube 3. With a fluid introduced into the expansion body 7 via the flow path 71, the expansion body 7 is expanded radially.

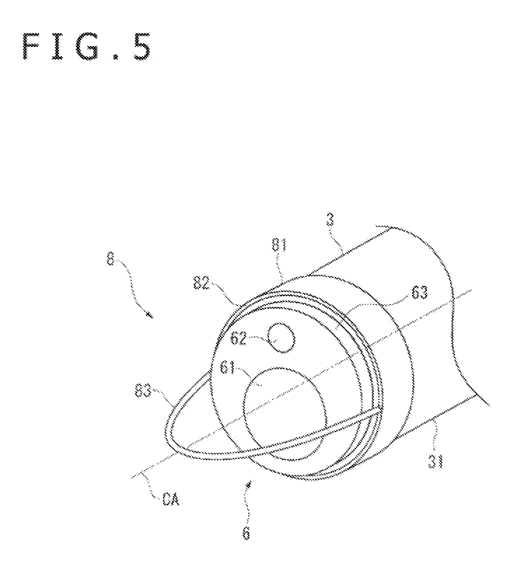

As shown in FIG. 5, the insertion guide part 8 is formed to be gradually decreased in diameter along the distal direction. Specifically, the insertion guide part 8 includes: a frame body 81 that is attached to a distal portion of the insertion tube 3; and a bridge portion 83 bridgingly provided between points of an end edge 82 of the frame body 81 in such a manner as to project toward the distal side of the insertion tube 3. The bridge portion 83 is composed of a single linear body which is flexible. The bridge portion 83 is bridgingly provided, in a curved shape, between two end points which are located symmetrically with respect to the center axis CA of the frame body 81. The insertion guide part 8, particularly the bridge portion 83, is configured by use of an elastic material, preferably an elastic metallic material; for example, it is preferably configured by use of a shape memory alloy that shows superelasticity in a use state. Such a shape memory alloy is preferably concretely composed of a nickel-titanium alloy. In FIG. 1, the positional relationship between the insertion guide part 8 and the insertion tube 3 is omitted. The insertion guide part 8 may be attached to an outer circumferential surface of a distal end of the insertion tube 3, or may be attached to an inner circumferential surface of the distal end. Further, the insertion guide part 8 may be embedded in the inside of the insertion tube 3, or may be firmly attached in the manner of being clamped between an outer circumferential surface of the insertion tube 3 and other member. The method for firmly attaching the insertion guide part 8 is not specifically restricted. The insertion guide part 8 may be firmly attached by an adhesive, brazing or the like, may be soldered, or may be firmly attached by caulking.

As shown in FIGS. 2A and 2B, the grasping portion 9 includes: a first operating portion 91 as a position fixture for fixing an advanced/retracted position of the endoscope 6 inside the insertion tube 3; and a second operating portion 92 for expanding or contracting the expansion body 7 and for maintaining an expanded state of the expansion body 7.

The first operating portion 91 holds the endoscope 6 inside the grasping portion 9. The first operating portion 91 is provided to be slidable toward the insertion tube 3, and is configured to be fixable at a position indicated by solid line and a position indicated by alternate long and short dash line in FIG. 2A. Specifically, when the first operating portion 91 is located in the position of solid line, the endoscope 6 has been inserted to such an extent that the imaging section 61 reaches the insertion guide part 8; on the other hand, when the first operating portion 91 is located in the position of alternate long and short dash line, the endoscope 6 has been retracted to the proximal side of the insertion tube 3 relative to the conveyance paths 4A and 4B and is fixed in the retracted position.

The second operating portion 92 is provided in an advanceable and retractable manner in a fluid introduction path 94 communicating with the flow path 71 for the expansion body 7, and is so configured that the expansion body 7 is expanded or contracted when the second operating portion 92 is advanced or retracted. Specifically, the second operating portion 92 includes: a rod 96 put into screw engagement with a threaded hole formed in a plugging member 95 for plugging the fluid introduction path 94; a sealing member 97 which is provided on one end side of the rod 96 inside the fluid introduction path 94 and which seals the fluid introduction path 94; and a knob part 98 provided on the other end side of the rod 96 in the outside of the fluid introduction path 94.

Now, the use procedure and operation of the medical treatment instrument 1 when the medical treatment instrument 1 is used as a sinusitis treatment instrument, as an example of use of the medical treatment instrument 1, will be described below.

First, in a state in which the endoscope 6 has been inserted to the distal end of the insertion section 2, the operator inserts the insertion section 2 into a nostril. In this instance, the insertion guide part 8 guides the insertion section 2, while forcing open the insertion route toward an outer circumference of the insertion tube 3 by the bridge portion 83.

Here, because the bridge portion 83 is a linear body, its thickness size as viewed from the insertion direction is small, so that the resistance at the time of insertion can be suppressed. In addition, because the bridge portion 83 is flexible, it can follow the shape of the insertion route through deformation. Therefore, the ability of the insertion guide part 8 to pass the insertion route can be enhanced, and, accordingly, the insertion section 2 can be inserted easily. Furthermore, because the bridge portion 83 is bridgingly provided in a curved shape, the bridge portion 83 would not locally press the inside of the living body, so that it can be prevented from damaging the tissue inside the nasal cavity.

Meanwhile, the operator inserts the insertion section 2 while checking the conditions in the insertion route, based on an image acquired by the endoscope 6. Here, because the bridge portion 83 is composed of a linear body and the area of projection of the bridge portion 83 in the insertion direction is small, the bridge portion 83 can be prevented from obstructing the viewing of the image acquired by the endoscope 6. In addition, even where a sticky matter or solid matter is present in the insertion route, the bridge portion 83 can split such a matter into pieces, so that adhesion of the sticky matter or solid matter to the bridge portion 83 can be restrained. Consequently, a sufficient field of view for imaging by the endoscope 6 can be secured.

When the expansion body 7 of the insertion section 2 inserted is guided to a natural ostium of a paranasal sinus stenosed due to sinusitis, the operator can operate the second operating portion 92 so as to introduce a fluid into the expansion body 7 and expand the expansion body 7, thereby expanding and treating the stenosed part of the natural ostium. It is to be noted that when the insertion section 2 is slightly retracted after contraction of the expansion body 7, it is possible to confirm that the once stenosed part has been expanded, based on the image acquired by the endoscope 6.

Thereafter, the operator inserts the insertion section 2 through the expanded natural ostium into the paranasal sinus, whereon it is possible to check the conditions inside the paranasal sinus, based on an image acquired by means of the endoscope 6. In the case where a fluid (such as snivel) or sticky matter is accumulated in the paranasal sinus, the first operating portion 91 is operated so as to retract the endoscope 6 in the insertion tube 3 to the proximal side relative to the conveyance paths 4A and 4B, after which the fluid or sticky matter can be sucked through the lumen 33 of the insertion tube 3 and the first conveyance path 4A. In this instance, because the thickness size of the bridge portion 83 as viewed from the insertion direction is small, the bridge portion 83 can be prevented from blocking the suction; accordingly, the suction can be carried out rapidly and assuredly.

On the other hand, cleaning of the inside of the paranasal sinus with a washing fluid such as physiological salt solution can also be performed, by introducing the washing fluid into the second conveyance path 4B. Attendant on this, the washing fluid flows in the insertion tube 3 to the proximal side relative to the conveyance paths 4A and 4B, so that cleaning of the imaging section 61 of the endoscope 6 in the retracted state can be conducted simultaneously with the cleaning of the inside of the paranasal sinus. In this instance, like in the case of the suction mentioned above, the bridge portion 83 can be prevented from blocking the suction, so that the cleaning can be carried out effectively.

Because the medical treatment instrument 1 has the insertion guide part 8 including the bridge portion 83 which is bridgingly provided between points of the frame body 81 mounted to an outer circumference of the distal end of the insertion tube 3, the insertion tube 3 can be guided while forcing open the insertion route toward the outer circumference of the insertion tube 3 by the bridge portion 83. Therefore, the distal end of the insertion tube 3 can be prevented from being caught in a living body. In addition, because the bridge portion 83 is composed of a flexible linear body, the thickness size of the bridge portion 83 as viewed from the insertion direction can be made small, and the bridge portion 83 can follow the shape of the insertion route through deformation. Therefore, the ability of the insertion guide part 8 to pass the insertion route can be enhanced, while suppressing the resistance at the time of insertion. Accordingly, insertion of the medical treatment instrument 1 can be facilitated.

Because the medical treatment instrument 1 is provided with the endoscope 6 for acquiring an image of the forward side in the insertion direction of the insertion guide part 8, image information on the insertion guide part 8 and the forward side of the insertion guide part 8 can be acquired. Therefore, the operator can operate the medical treatment instrument 1 while checking the image information acquired. In addition, because the bridge portion 83 of the insertion guide part 8 is composed of a linear body, the area of projection in the insertion direction of the bridge portion 83 can be made small. Therefore, the bridge portion 83 can be prevented from obstructing the viewing of the image acquired by the endoscope 6. Accordingly, a sufficient field of view for the endoscope 6 can be secured.

Further, because the fluid conveyance path 10 opening toward the insertion guide part 8 is provided, it is possible to convey a fluid from the inside of a living body to the outside or to convey a fluid from the outside into the inside of the living body, through the fluid conveyance path 10. Because the bridge portion 83 of the insertion guide part 8 is composed of a linear body, the bridge portion 83 can be prevented from blocking the conveyance of a fluid.

In addition, because the expansion body 7 can be assuredly guided into a stenosed part in a living body by the insertion guide part 8, the stenosed part can be expanded and treated by expansion of the expansion body 7.

Furthermore, because the medical treatment instrument 1 has the insertion guide part 8, the medical treatment instrument 1 can be assuredly inserted into a paranasal sinus or into a natural ostium through which the paranasal sinus and a nasal cavity communicate with each other. Accordingly, a treatment necessary for the sinusitis treatment can be carried out, without performing any surgical procedure.

Now, other embodiments of the present invention will be described below. Here, medical treatment instruments 1 to be used in a second and latter embodiments are each different from that used in the first embodiment in the shape of the insertion guide part 8.

### Second Embodiment

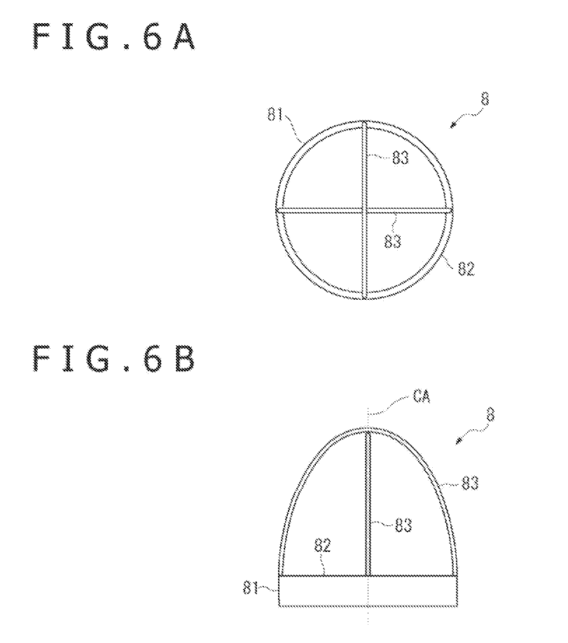

A second embodiment of the present invention will be described, based on FIGS. 6A and 6B.

In a medical treatment instrument 1 according to this embodiment, as shown in FIGS. 6A and 6B, an insertion guide part 8 includes a frame body 81, and two bridge portions 83 which are each composed of a single linear body and which are provided in different positions of an end edge 82 of the frame body 81. Here, the bridge portions 83 are each bridgingly provided between points of the end edge 82 of the frame body 81 so that they intersect with each other on a center axis CA of the frame body 81. Distal ends in the projection direction of the bridge portions 83 are not fixed to each other.

According to this embodiment, the following effects are obtained, in addition to the effects of the first embodiment.

Because the bridge portions 83 are provided in different positions of the end edge 82 of the frame body 81, the distal end of an insertion tube 3 can be guided, and the distal end of the insertion tube 3 can be prevented from being caught. Further, because the distal ends in the projection direction of the bridge portions 83 are not fixed to each other, the bridge portions 83 can be deformed in a flexible manner. Consequently, insertion of the medical treatment instrument 1 can be further facilitated.

### Third Embodiment

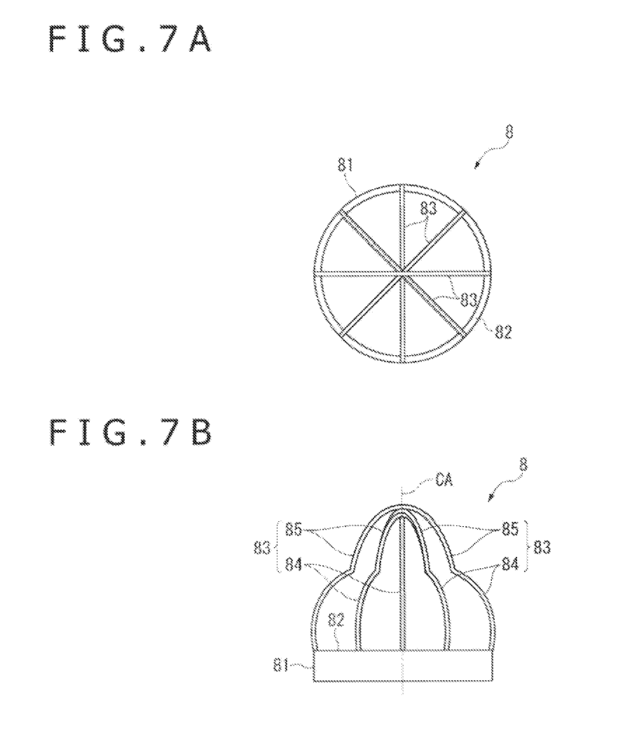

Now, a third embodiment of the present invention will be described below, based on FIGS. 7A and 7B.

In a medical treatment instrument 1 according to this embodiment, as shown in FIGS. 7A and 7B, an insertion guide part 8 includes a frame body 81, and four bridge portions 83 which are each composed of a single linear body and which are arranged, at regular intervals, in different positions of an end edge 82 of the frame body 81. Each of the bridge portions 83 includes: a first curved section 84 curved from the frame body 81 toward a center axis CA of the frame body 81; and a second curved section 85 curved from the first curved section 84 toward the center axis CA with a curvature smaller than the curvature of the first curved section 84. Thus, the insertion guide part 8 has a configuration in which the bridge portions 83 are each bridgingly provided between points of the end edge 82 of the frame body 81 so that they intersect each other on the center axis CA of the frame body 81, and in which the curved portions 84 and 85 of each bridge portion 83 ensures that the diameter of the insertion guide part 8 decreases in two steps toward the distal end of the insertion guide part 8. It is to be noted that the distal ends in the projection direction of the bridge portions 83 are not fixed to one another.

According to this embodiment, the following effects are obtained, in addition to the effects of the first embodiment.

Because the diameter of the insertion guide part 8 decreases in multiple steps toward the distal end of the insertion guide part 8, the insertion guide part 8 has a kind of link structure whose centers are determined by junction points between the steps of the bridge portions 83. Therefore, a degree of rigidity of the bridge portion 83 necessary for guiding can be secured, while maintaining the follow-up performance of the bridge portions 83 in relation to an insertion route. In addition, because the degree of decrease in diameter can be set on the basis of each of the steps, the projection shape of the insertion guide part 8 can be set according to the use of the medical treatment instrument 1 or the insertion route. Accordingly, insertion of the medical treatment instrument 1 can be further facilitated.

### Fourth Embodiment

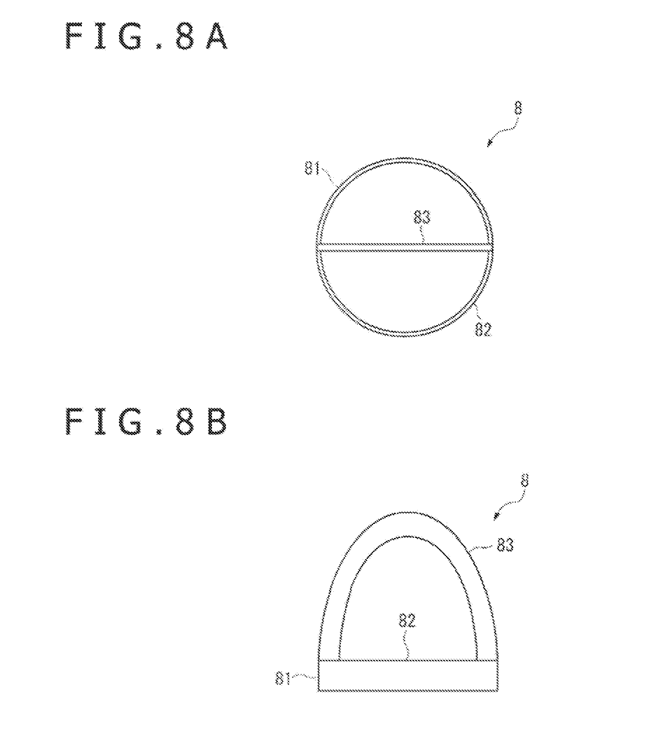

Now, a fourth embodiment of the present invention will be described below, based on FIGS. 8A and 8B.

In a medical treatment instrument 1 according to this embodiment, as shown in FIGS. 8A and 8B, an insertion guide part 8 includes a frame body 81, and a single bridge portion 83 composed of a band-shaped body. Here, the bridge portion 83 is bridgingly provided, in a curved shape, between points of an end edge 82 so that an end portion in the width direction of the band-shaped body projects toward the distal side of an insertion tube 3. Other aspects of the configuration of the insertion guide part 8 are the same as in the first embodiment, and, therefore, description of the other aspects are omitted here.

According to this embodiment, the following effects are obtained, in addition to the effects of the first embodiment.

Because the bridge portion 83 is composed of a band-shaped body and an end portion in the width direction of the band-shaped body projects toward the distal side, a degree of rigidity of the bridge portion 83 necessary for guiding can be secured, while keeping the bridge portion 83 thin as viewed from the insertion direction. Therefore, the bridge portion 83 can be prevented from being collapsed toward the proximal side of the insertion tube 3, attendant on insertion. Accordingly, guiding by the insertion guide part 8 can be performed reliably.

### Fifth Embodiment

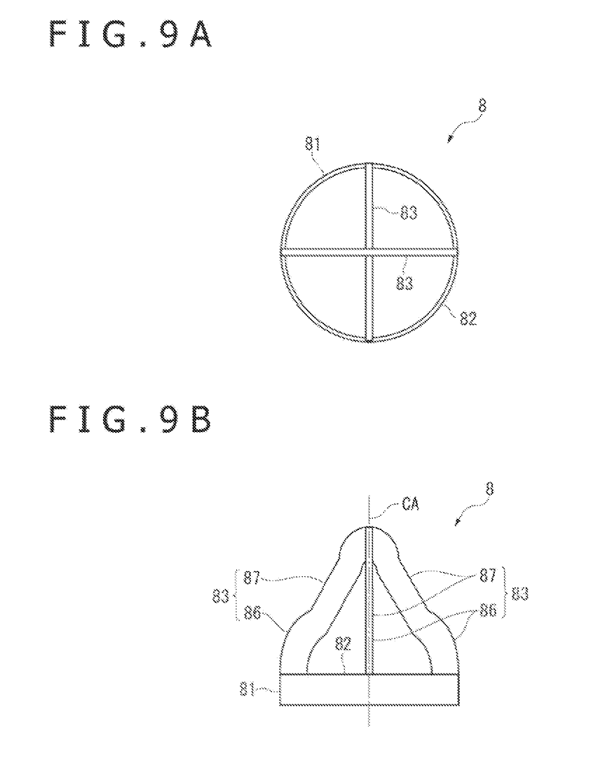

Now, a fifth embodiment of the present invention will be described below, based on FIGS. 9A and 9B.

In a medical treatment instrument 1 according to this embodiment, as shown in FIGS. 9A and 9B, an insertion guide part 8 includes a frame body 81, and two bridge portions 83 which are each composed of a band-shaped body and each of which is bridgingly provided between points of an end edge 82 so that an end portion in the width direction of the band-shaped body projects toward the distal side of an insertion tube 3. Each bridge portion 83 includes: a curved portion 86 curved from the frame body 81 toward a center axis CA of the frame body 81; and a straight portion 87 extending straight from the curved portion 86 toward the center axis CA. Thus, the curved portions 86 and the straight portions 87 of the bridge portions 83 ensure that the diameter of the insertion guide part 8 decreases in two steps along the distal direction. It is to be noted that the distal ends in the projection direction of the bridge portions 83 are not fixed to each other. Other aspects of the configuration of the insertion guide part 8 are the same as in the second embodiment, and, therefore, descriptions of the other aspects are omitted here.

It is to be noted that the present invention is not to be restricted to the aforementioned embodiments, and the invention includes those modifications, improvements and the like which are within such a scope that the object of the invention can be achieved.

For instance, while the insertion guide part 8 is provided at the distal end of the insertion tube 3 for accommodating the endoscope 6 in the above embodiments, this is not restrictive. The insertion guide part 8 can be attached to any part of the medical treatment instrument insofar as the part is in a distal portion inserted into a living body. For example, the insertion guide part 8 can be used also for a medical treatment instrument such as an endoscope, a guiding catheter, a balloon catheter, etc. In addition, the sectional shape of the insertion guide part 8 and the sectional shape of the elongated body to which the insertion guide part 8 is attached are not restricted to a true circle, and may, for example, be an ellipse, a polygon, or the like.

In addition, while the diameter of the insertion guide part 8 is decreased in two steps in the third and fifth embodiments, the diameter may be decreased in three or more steps.

Furthermore, while the insertion guide part 8 has the bridge portions 83 each composed of a linear body or band-shaped body in the second, third and fifth embodiments, a bridge portion or portions 83 each composed of a linear body and a bridge portion or portions 83 each composed of a band-shaped body may be provided in combination.

While the expansion body 7 is provided at an outer circumference of the insertion tube 3 in the above embodiments, the medical treatment instrument 1 may be configured without providing any such expansion body 7. Specifically, a configuration may be adopted in which the medical treatment instrument 1 includes a balloon catheter, and the balloon catheter can be inserted into and withdrawn from the lumen 33 via the first conveyance path 4A or the second conveyance path 4B. Thus, it is not indispensable that the conveyance path in the insertion section 2 be configured as the fluid conveyance path 10; in other words, the conveyance path in the insertion section 2 may be configured as a conveyance path through which to convey such an object to be conveyed as a balloon catheter or a guide wire.

While the retracted position of the endoscope 6 in the insertion tube 3 can be fixed by the first operation portion 91 in the above embodiments, this is not restrictive of the means of fixing the retracted position of the endoscope 6. For instance, of the endoscope 6, the distal portion where the imaging section 61 is provided may be enlarged in diameter, or a portion on the proximal side may be set smaller than the distal portion in diameter, whereby movement of the distal portion can be restricted by the valve body 5, and the retracted position of the endoscope 6 can be fixed.

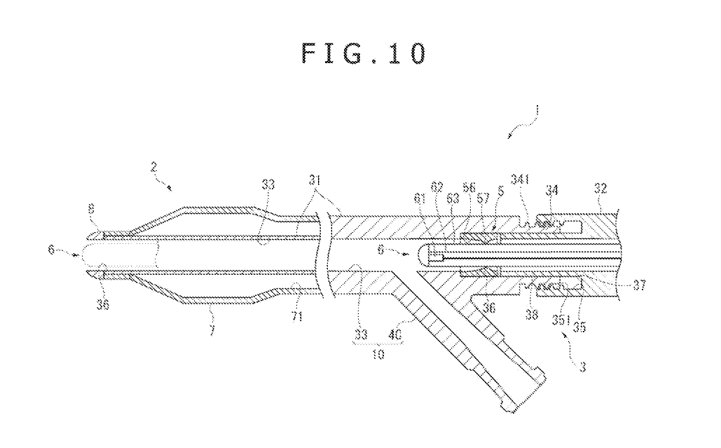

In addition, the structure of sealing of the insertion tube 3 by the valve body 5 is not restricted to those in the above embodiments. For instance, a structure as shown in FIG. 10 may be adopted.

In FIG. 10, at an intermediate portion of a distal tube 31, a conveyance path 4C branched in a Y shape from the distal tube 31 is provided in communication with a lumen 33 of the distal tube 31, and a proximal portion 34 of the distal tube 31 is formed with a male screw 341 at an outer circumference thereof. At an end face of the proximal portion 34, a recess 36 greater in inside diameter than the lumen 33 is provided, and the valve body 5 is accommodated in the recess 36.

A recess 35 in a proximal tube 32 is formed at an inner circumference thereof with a female screw 351 for screw engagement with the male screw 341 of the distal tube 31. In addition, at the recess 35, there is provided a projecting portion 38 which projects in the axial direction of the proximal tube 32 from a bottom surface 37 of the recess 35.

In FIG. 10, the valve body 5 is configured by use of an elastic material such as rubber or resin. The valve body 5 is provided with a penetrating path 56 penetrating therethrough from an end face 51 on one side to an end face 53 on the other side, and with a diametrically reduced portion 57 where the diameter of the penetrating path 56 is partly reduced.

In this medical treatment instrument 1, when the distal tube 31 and the proximal tube 32 are rotated relative to each other, starting from the state as shown in FIG. 10, to thereby bring the distal tube 31 and the proximal tube 32 toward each other along the axial direction, the valve body 5 is pushed in the axial direction by the projecting portion 38 of the proximal tube 32. This results in that the valve body 5 is deformed, the penetrating path 56 as a whole is reduced in diameter, and the lumen 33 of the insertion tube 3 is held liquid-tight by the diametrically reduced portion 57 of the penetrating path 56.

While the medical treatment instrument 1 is used for observation of the inside of a paranasal sinus and/or treatment of sinusitis in the above embodiments, the medical treatment instrument 1 may be used for observation or treatment of other part in a living body.

The present invention is applicable not only to medical treatment instruments for diagnosis or therapy of a paranasal sinus but also to medical treatment instruments for other diagnoses or therapies that are not attended by a surgical procedure.

What is claimed is:

1. A medical treatment instrument comprising:
a tube-shaped elongated main body; and
an insertion guide part mounted to a distal end of the elongated main body, wherein the insertion guide part comprises:
a frame body mounted to a distal portion of the elongated main body; and
a flexible bridge portion which is disposed so as to bridge points of an end edge of the frame body, the flexible bridge portion comprising a linear body or band-shaped body projecting in a distal direction beyond the elongated main body.

2. The medical treatment instrument according to claim 1, wherein the medical treatment instrument comprises a plurality of bridge portions that are provided at different positions of the end edge.

3. The medical treatment instrument according to claim 2, wherein each of the plurality of bridge portions comprises a distal end, and the distal ends of each of the plurality of bridge portions are not fixed to each other.

4. The medical treatment instrument according to claim 1, wherein the bridge portion is composed of a band-shaped body, and an end portion of the band-shaped body projects in the distal direction beyond the elongated main body.

5. The medical treatment instrument according to claim 1, wherein the insertion guide part decreases in diameter in multiple steps along the distal direction.

6. The medical treatment instrument according to claim 3, wherein the insertion guide part decreases in diameter in multiple steps along the distal direction.

7. The medical treatment instrument according to claim 1, further comprising an image information acquisition section which is provided inside the elongated main body and which acquires an image of a forward side in an insertion direction of the insertion guide part.

8. The medical treatment instrument according to claim 1, further comprising a fluid conveyance path which is provided in the elongated main body and which is open toward the insertion guide part.

9. The medical treatment instrument according to claim 1, further comprising an expansion body which is provided at an outer circumference on a distal side of the elongated main body and which is expandable outward in a radial direction of the elongated main body.

10. The medical treatment instrument according to claim 1, wherein the medical treatment instrument is a sinusitis treatment instrument.
